(19) United States
(12) Patent Application Publication
(10) Pub. No.: US 2020/0315855 A1
(43) Pub. Date: Oct. 8, 2020

(54) REHABILITATIVE TAPE SPLITTER AND METHOD OF USING THE SAME

(71) Applicant: Bradley Shallow, Port Orange, FL (US)

(72) Inventor: Bradley Shallow, Port Orange, FL (US)

(21) Appl. No.: 16/954,144

(22) PCT Filed: Dec. 20, 2018
(86) PCT No.: PCT/US2018/066958

§ 371 Date: Jun. 16, 2020

Related U.S. Application Data
(60) Provisional application No. 62/608,430, filed on Dec. 20, 2017.

(51) Int. Cl. A61F 13/00; A61F 13/02; B26D 1/03; B26D 1/09; B26D 5/10

(52) U.S. Cl. CPC A61F 13/00076; A61F 13/0273; B26D 5/10; B26D 1/095; B26D 1/035

(57) ABSTRACT

A hand-held and operated device for splitting rehabilitative elastic tape into segments and rounding corners of the rehabilitative elastic tape segments and method of use is disclosed. The device comprises a feed roll of rehabilitative elastic tape, and a housing. The housing is comprised of a vertical cutting mechanism and a cutting die punch mechanism operably coupled in tandem. There are a plurality of actuator buttons operably coupled to and aligned with the blades for setting the blades to either a cutting position or a non-cutting position. The cutting die punch mechanism is comprised of a pair of mating tools, a die punch and a cutting die. The die punch is adapted to be moved into the cutting die by hand and attain the closest possible mating of said members for cutting or forming the rounded corners of a tape segment when the tape is fed into the punching mechanism.

### I. FIELD OF THE INVENTION

The present invention relates generally to devices and methods used to cut rehabilitative tape. More specifically, the present invention relates to devices and methods to cut rehabilitative tap into segments with rounded corners.

### II. BACKGROUND

Rehabilitative tape is used in physical medicine by rehabilitation doctors and physical therapists to tape a limb to increase blood flow to that limb, allowing for better performance and faster recovery from injury. Normally, a physical medicine by rehabilitation doctors and physical therapists would have to split the tape into multiple sections and round the corners of the tape by hand, a time-consuming effort for both the practitioner and the patient. One objective of the present invention is to provide an apparatus for splitting rehabilitative tape into segments with rounded corners for use in improving blood flow to a patient's limbs, in one convenient step. Therefore, there is a present need for an apparatus used to split rehabilitative tape into segments with rounded corners for use in improving blood flow to a patient's limbs.

### III. SUMMARY OF THE INVENTION

A first aspect of the present invention provides a hand-held and operated device for splitting rehabilitative elastic tape into segments and rounding corners of the rehabilitative elastic tape segments. The device is comprised of a feed roll of rehabilitative elastic tape, having a substantially uniform thickness and width, and a housing. The housing is comprised of a vertical cutting mechanism and a cutting die punch mechanism operably coupled in tandem. The vertical cutting mechanism is comprised of a plurality of blades. Each blade has a cutting edge, and the cutting edge of the blade extends in the opposite direction of the relative motion between the tape and the blades when the tape is fed into the vertical cutting mechanism. The angle between the cutting edge of the planar blades and the surface of the tape is between 15 and 75 degrees. There are a plurality of actuator buttons operably coupled to and aligned with the blades for setting the blades to either a cutting position or a non-cutting position, such that when the blades are in the cutting position the cutting edge is in contact with surface of the tape. The cutting die punch mechanism is comprised of a pair of mating tools, a die punch and a cutting die. The die punch is provided with a fixed punching blade member and the cutting die with a flexible cutting blade member, with the fixed punching blade member and said flexible cutting blade member each having a continuous cutting edge. The die punch is adapted to be moved into the cutting die by hand and attain the closest possible mating of said members for cutting or forming the rounded corners of a tape segment when the tape is fed into the punching mechanism.

A second aspect of the present invention provides a hand-held and operated device for splitting rehabilitative elastic tape into segments and rounding corners of the rehabilitative elastic tape segments. The device is comprised of a feed roll of rehabilitative elastic tape, having a substantially uniform thickness and width, and a housing. The housing is comprised of a vertical cutting mechanism and a cutting die punch mechanism operably coupled in tandem. The vertical cutting mechanism has a plurality of blades, where each blade has a cutting edge. The vertical cutting mechanism has a plurality of actuator buttons operably coupled to and aligned with the blades for setting the blades to either a cutting position or a non-cutting position, such that when the blades are in the cutting position the cutting edge is in contact with surface of the tape. The cutting implements in the cutting position splits the rehabilitative tape into two or more sections. The cutting die punch mechanism is comprised of a pair of mating tools, a die punch and a cutting die. The die punch is provided with a fixed punching blade member and the cutting die with a flexible cutting blade member, with the fixed punching blade member and said flexible cutting blade member each having a continuous cutting edge. The die punch is adapted to be moved into the cutting die by hand and attain the closest possible mating of said members for cutting or forming the rounded corners of a tape segment when the tape is fed into the punching mechanism.

A third aspect of the present invention comprises a method of cutting rehabilitative tape. The method includes a hand-held and operated device for splitting rehabilitative elastic tape into segments and rounding corners of the rehabilitative elastic tape segments. The device has a feed roll of rehabilitative elastic tape, having a substantially uniform thickness and width, and a housing. The housing is made up of a vertical cutting mechanism and a cutting die punch mechanism operably coupled in tandem. The vertical cutting mechanism has a plurality of blades, where each blade has a cutting edge, and a plurality of actuator buttons. The actuator buttons are operably coupled to and aligned with the blades for setting the blades to either a cutting position or a non-cutting position, such that when the blades are in the cutting position the cutting edge is in contact with surface of the tape. The cutting implements in the cutting position split the rehabilitative tape into two or more sections. The cutting die punch mechanism is comprised of a pair of mating tools, a die punch and a cutting die, characterized in that said die punch is provided with a fixed punching blade member and said cutting die with a flexible cutting blade member, said fixed punching blade member and said flexible cutting blade member each having a continuous cutting edge, said die punch being adapted to be moved to operably couple said cutting die by hand, attaining a closest possible mating of said members for cutting or forming the rounded corners of a tape segment when the tape is fed into the punching mechanism. The rehabilitative tape has a first end, a second end, a splitting point and a remaining portion. The first step of the method is stretching a length of rehabilitative tape from the tape roll, placing the first end in the cutting die punch mechanism. Then, the second step of the method is activating the cutting die punch mechanism, and rounding the corners of the first end of the rehabilitative tape. Then, the third step of the method is pulling the rehabilitative tape to the splitting point, aligned with the plurality of blades. The fourth step of the method is setting one or more of the plurality of blades from the non-cutting position to the cutting position so that the exposed length of rehabilitative tape is split into two or more sections. The fifth step of the method is pulling the length of rehabilitative tape by hand until the split rehabilitative tape until the second end is aligned with the cutting die punch mechanism. The sixth step of the method is activating the cutting die punch mechanism, rounding the corners of the second end of the rehabilitative tape and separating it from the remaining portion of the rehabilitative tape roll. The seventh step of the method is pulling the tape until the second end has been split into two or more sections by the plurality of blades. The eighth step of the method is retracting the plurality of blades. The ninth step of the method is applying the improved rehabilitative tape to a patient to increase blood circulation and support muscles in an affected area.

A fourth aspect of the present invention is a method of splitting rehabilitative elastic tape into sections (0-60). The method includes providing a hand-held and operated device for splitting rehabilitative elastic tape into sections (39) and rounding corners (35) of the rehabilitative elastic tape sections. The device includes a feed roll of rehabilitative elastic tape, having a substantially uniform thickness and width. In addition the device includes a housing. The housing includes a vertical cutting mechanism and a cutting die punch mechanism operably coupled in tandem. The vertical cutting mechanism includes a plurality of blades. Each blade has a cutting edge, and a plurality of actuator buttons operably coupled to and aligned with the blades for setting the blades to either a cutting position or a non-cutting position, such that when the blades are in the cutting position the cutting edge is in contact with a surface of the tape. The cutting implements in the cutting position split the rehabilitative tape into two or more sections (39). The cutting die punch mechanism includes a pair of mating tools, a die punch and a cutting die, characterized in that said die punch is provided with a fixed punching blade member and said cutting die with a flexible cutting blade member. The fixed punching blade member and said flexible cutting blade member each have a continuous cutting edge. The die punch is adapted to be moved to operably couple said cutting die by hand, attaining a closest possible mating of said members for cutting or forming the rounded corners of a tape segment when the tape is fed into the punching mechanism. The rehabilitative tape has a first end (45), a second end (47), a splitting point (49) of tape (4) and a remaining portion (51) of tape (4). In a second step the first end (45) of the rehabilitative tape is inserted into the cutting die punch mechanism (9). In a third step the cutting die punch mechanism (9) is activated, rounding the corners of the first end of the rehabilitative tape (4). In a fourth step, a first end (45) of the rehabilitative elastic tape is pushed into the cutting die punch mechanism (9) and the vertical cutting mechanism (7), until the first end (45) extends out of the vertical cutting mechanism (7). In a fifth step, the rehabilitative tape is pulled in the direction of arrow (59) to the splitting point (49), and aligned with the plurality of blades (11) until the second end (47) of the split rehabilitative tape is coextensive with the line 49-49, demarking the beginning of the split portion (149) between lines 49-49 and 60-60 of the segment of split tape (87). In a sixth step, the rehabilitative tape (43) is pulled by hand in the direction of the arrow (63) until the first end (45) of the rehabilitative tape is coextensive with the line 60-60, demarking the end of the split portion 160 between lines 60-60 and of the segment of split tape (87). In a seventh step, one or more of the plurality of blades (11) is set from the non-cutting position 23 to the cutting position 21 so that the exposed length 43 of rehabilitative tape is split into two or more sections 39. In an eighth step, the rehabilitative tape (43) is pulled by hand in the direction of the arrow (63) until the first end (45) of the rehabilitative tape is coextensive with the line 60-60, demarking the end of the split portion (160) between lines 60-60 and of the segment of split tape (87). In a ninth step, the cutting die punch mechanism (9), is activated, rounding the corners (35) of the second end of the rehabilitative tape and separating it from the remaining portion (51) of the rehabilitative tape roll. In a tenth step, the tape is pulled until the second end (47) has been split into two or more sections by the plurality of blades. In an eleventh step, the plurality of blades (11) are retracted. In a twelfth step, the improved rehabilitative tape is applied to a patient to increase blood circulation and support muscles in an affected area. In a thirteenth step, for future uses of the rehabilitative tape from the same roll, a first end (45) of the rehabilitative elastic tape currently in cutting die punch mechanism (9) is fed into the vertical cutting mechanism (7), until the first end (45) extends out of the vertical cutting mechanism (7). In a fourteenth step, steps 59-71 are repeated.

### BRIEF DESCRIPTION OF THE DRAWINGS

The features of the invention are set forth in the appended claims. The invention itself, however, will be best understood by reference to the following detailed description of an illustrative embodiment when read in conjunction with the accompanying drawings, wherein:

FIG. 1 depicts a cross-sectional side view of the rehabilitative tape splitter, in accordance with embodiments of the present invention;

FIGS. 2A, 2B depict a cross-sectional front view of the rehabilitative tape splitter and an unsplit segment of the tape, in accordance with embodiments of the present invention;

FIGS. 3A, 3B and 3C depict a cross-sectional front view of the rehabilitative tape splitter and an split segment of the tape, in accordance with embodiments of the present invention;

FIG. 4 depicts a flow diagram of a method of splitting rehabilitative tape, in accordance with embodiments of the present invention;

FIG. 5A and FIG. 5B. depicts a cross-sectional side view of the rehabilitative tape splitter, in accordance with embodiments of the present invention;

FIGS. 6A and 6B depicts a cross-sectional front view of the rehabilitative tape splitter, in accordance with embodiments of the present invention;

FIGS. 7A and 7B depict a cross-sectional front view of the rehabilitative tape splitter, in accordance with embodiments of the present invention;

FIGS. 8A and 8B depict a cross-sectional view of the rehabilitative tape splitter, in accordance with embodiments of the present invention; and

FIG. 9 depicts a flow diagram of a method of splitting rehabilitative tape, in accordance with embodiments of the present invention.

### DETAILED DESCRIPTION OF THE EMBODIMENTS OF THE INVENTION

### Definitions: Fixed

Hereinafter, unless otherwise defined, the term “a fixed blade” refers to a blade that is firmly attached to the adjacent mechanism such that if the mechanism moves, it moves, where it cannot move independently of the mechanism although it can still be disassembled and replaced.

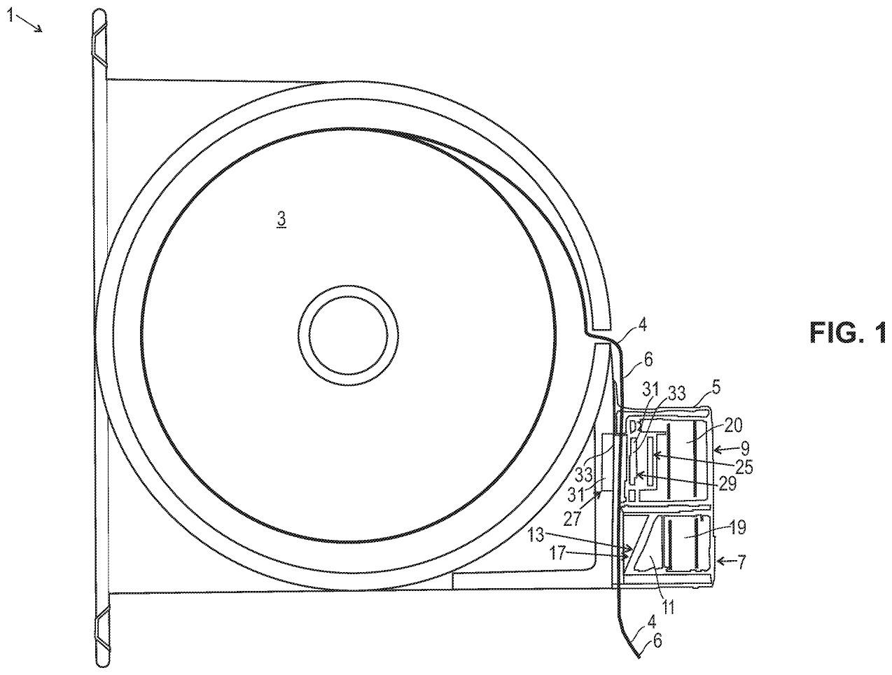

FIG. 1 depicts a cross-sectional side view of the rehabilitative tape splitter. A first aspect of the present invention provides a hand-held and operated device 1 for splitting rehabilitative elastic tape into segments and rounding corners of the rehabilitative elastic tape segments. The device is comprised of a feed roll 3 of rehabilitative elastic tape 4, having a substantially uniform thickness and width, and a housing 5.

In FIG. 1, flexible cutting blade member 31 and its continuous cutting edge 33 reference the same place, since 33 is the continuous cutting edge of cutting die member 31. Flexible cutting blade member 31 belongs with cutting die 27 and appears above and below the surface 6 of tape 4.

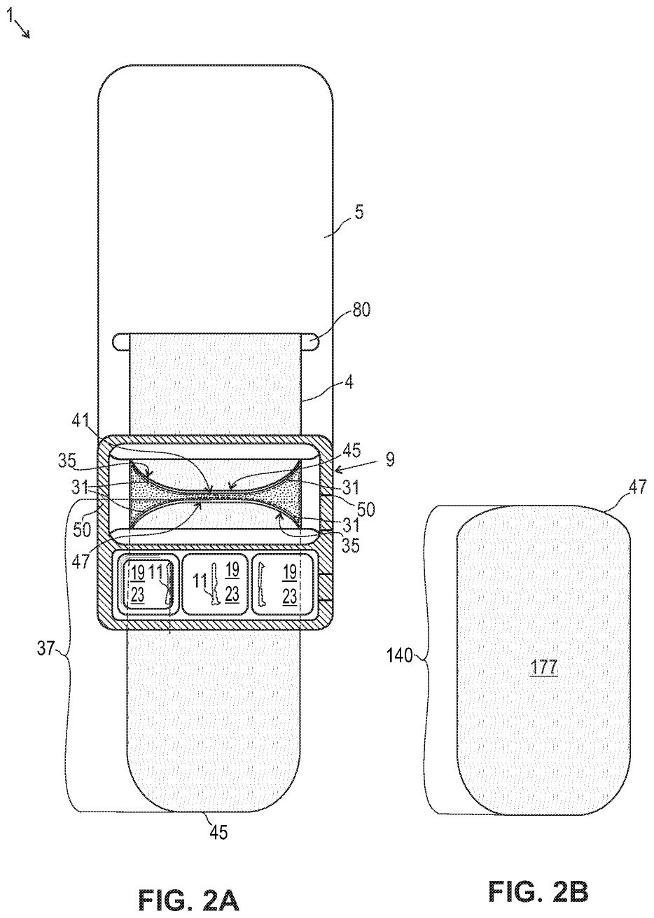

FIG. 2 depicts a cross-sectional front view of the rehabilitative tape splitter, in accordance with embodiments of the present invention. The housing 5 is comprised of a vertical cutting mechanism 7 and a cutting die punch mechanism 9 operably coupled in tandem. The vertical cutting mechanism is comprised of a plurality of blades 11. Each blade has a cutting edge 13, and the cutting edge of the blade extends in the opposite direction of the relative motion between the tape 4 and the blades 11 when the tape is fed into the vertical cutting mechanism 7. The angle 17 between the cutting edge of the planar blades 11 and the surface of the tape 4 is between 15° F. and 75° F. There are a plurality of actuator buttons 19 operably coupled to and aligned with the blades 11 for setting the blades 11 to either a cutting position 21 or a non-cutting position 23, such that when the blades 11 are in the cutting position 21 the cutting edge 13 is in contact with surface of the tape 4. The cutting die punch mechanism 9 is comprised of an actuator 20, a pair of mating tools, a die punch 25 and a cutting die 27. The die punch 25 is provided with a fixed punching blade member 29 and the cutting die 27 with a flexible cutting blade member 31, with the fixed punching blade member 29 and said flexible cutting blade member 31 each having a continuous cutting edge 33. The die punch is adapted to be moved into the cutting die by hand and attain the closest possible mating of said members 29, 31 for cutting or forming the rounded corners 35 of a tape segment 37 when the tape is fed into the punching mechanism.

A second aspect of the present invention provides a hand-held and operated device 1 for splitting rehabilitative elastic tape into segments and rounding corners of the rehabilitative elastic tape segments. The device is comprised of a feed roll 3 of rehabilitative elastic tape 4, having a substantially uniform thickness and width, and a housing 5. The housing 5 is comprised of a vertical cutting mechanism 7 and a cutting die punch mechanism 9 operably coupled in tandem. The vertical cutting mechanism is comprised of a plurality of blades 11. Each blade has a cutting edge 13. There are a plurality of actuator buttons 19 operably coupled to and aligned with the blades 11 for setting the blades 11 to either a cutting position 21 or a non-cutting position 23, such that when the blades 11 are in the cutting position 21 the cutting edge 13 is in contact with surface of the tape 4. The cutting implements in the cutting position 21 splits the rehabilitative tape 4 into two or more sections 39. The cutting die punch mechanism 9 is comprised of a pair of mating tools, a die punch 25 and a cutting die 27. The die punch 25 is provided with a fixed punching blade member 29 and the cutting die 27 with a flexible cutting blade member 31, with the fixed punching blade member 29 and said flexible cutting blade member 31 each having a continuous cutting edge 33. The die punch is adapted to be moved into the cutting die by hand and attain the closest possible mating of said members 29, 31 for cutting or forming the rounded corners 35 of a tape segment 37 when the tape is fed into the punching mechanism.

In one embodiment, the die punch is in the shape of two oppositely aligned half circles 41 connected at the point 90 degrees along their arc. This allows a single die punch to round the corners of incoming and outgoing pieces of tape.

In one embodiment, there is a plurality of die punches 25, each one designed to round the corners of a specific section of tape. These could section the tape into proportions such as a whole piece of tape, a half piece of tape, a third of a piece of tape, a fourth of a piece of tape, a sixth of a piece of tape, and a twelfth of a piece of tape. Typically the rehabilitative tape 4 is Kinesio Tape or the like.

In one embodiment, the apparatus is operably coupleable to a flat surface. In another embodiment, the apparatus is removably coupleable to a flat surface.

In one embodiment, an exposed length 43 of rehabilitative tape is demarcated to an advantageous length for increasing blood circulation in an affected area prior to cutting and rounding the corners.

In an embodiment, the plurality of blades 11, cutting die 7, punch mechanism 9, die punch 25, or cutting die 27 are replaceably disposable, as shown in FIG. 1 as a cutting die punch mechanism cartridge 94 and a vertical cutting mechanism cartridge 96.

In an embodiment, the disposable cutting implements are selected from the group consisting of single edge razor blades, snap-off blades, and utility knife blades.

In an embodiment, the cutting punch 9 is removable for cleaning.

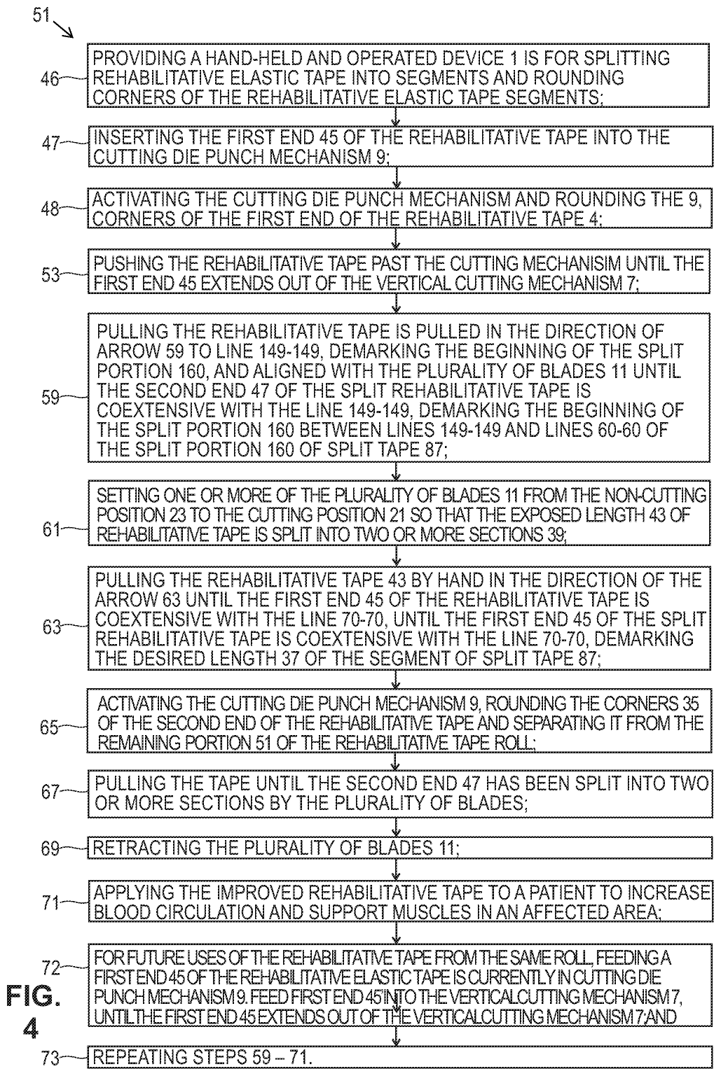

A third aspect of the present invention comprises a method 51 of cutting rehabilitative tape 4, as shown in FIG. 4. The method includes a hand-held and operated device 1 for splitting rehabilitative elastic tape into segments and rounding corners of the rehabilitative elastic tape segments. The device is comprised of a feed roll 3 of rehabilitative elastic tape 4, having a substantially uniform thickness and width, and a housing 5. The housing 5 is comprised of a vertical cutting mechanism 7 and a cutting die punch mechanism 9 operably coupled in tandem. The vertical cutting mechanism is comprised of a plurality of blades 11. Each blade has a cutting edge 13. There are a plurality of actuator buttons 19 operably coupled to and aligned with the blades 11 for setting the blades 11 to either a cutting position 21 or a non-cutting position 23, such that when the blades 11 are in the cutting position 21 the cutting edge 13 is in contact with surface of the tape 4. The cutting implements in the cutting position 21 splits the rehabilitative tape 4 into two or more sections 39. The cutting die punch mechanism 9 is comprised of a pair of mating tools, a die punch 25 and a cutting die 27. The die punch 25 is provided with a fixed punching blade member 29 and the cutting die 27 with a flexible cutting blade member 31, with the fixed punching blade member 29 and said flexible cutting blade member 31 each having a continuous cutting edge 33. The die punch is adapted to be moved into the cutting die by hand and attain the closest possible mating of said members 29, 31 for cutting or forming the rounded corners 35 of a tape segment 37 when the tape is fed into the punching mechanism.

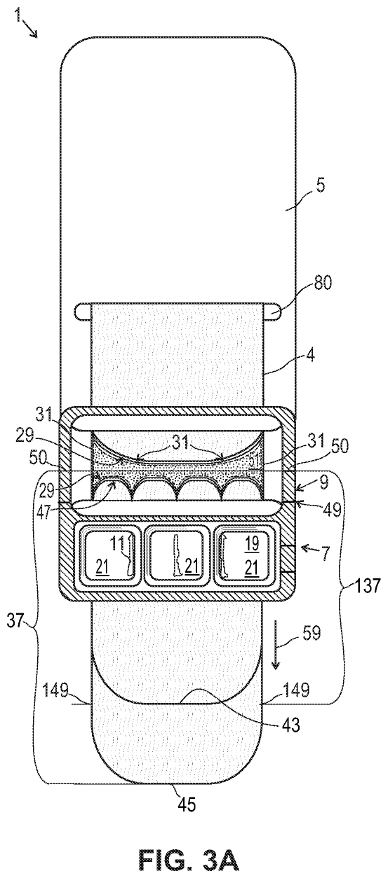

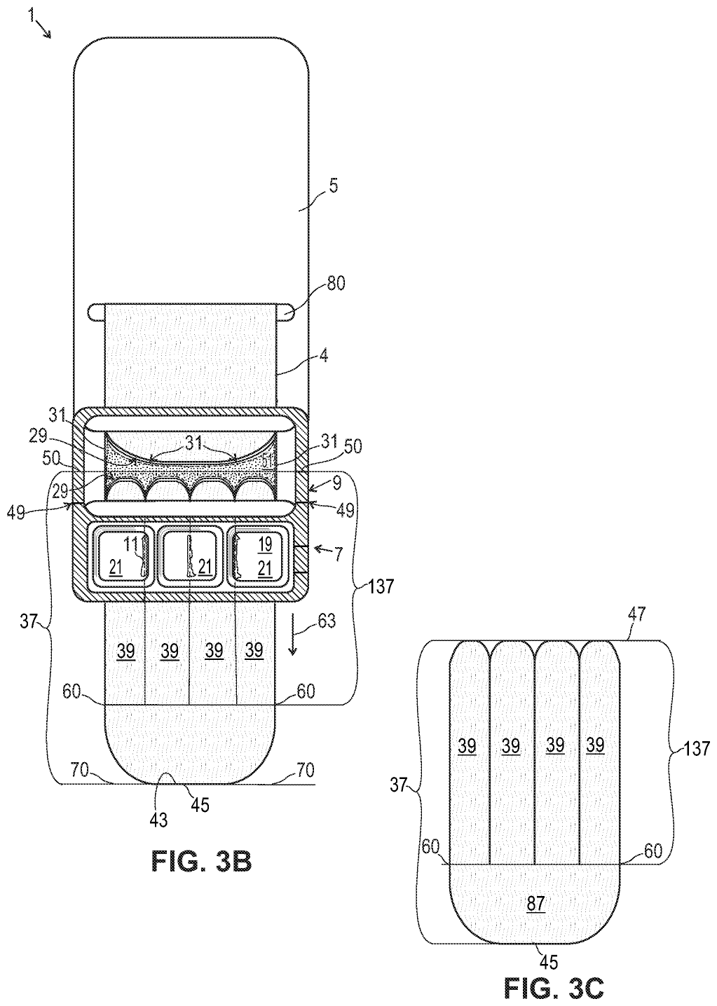

FIG. 4 depicts a flow diagram of a method 51 of cutting rehabilitative tape. An illustrative example of forming of a segment of split tape 87, by cutting rehabilitative tape, is shown in FIGS. 3A-3C, infra, and described in associated text herein.

In a first step 46 of the method 51, a hand-held and operated device 1 is provided for splitting rehabilitative elastic tape into segments and rounding corners of the rehabilitative elastic tape segments. In a second step 47 of the method 51, the first end 45 of the rehabilitative tape is inserted into the cutting die punch mechanism 9. In a third step 48 of the method 51, the cutting die punch mechanism 9 is activated, rounding corners of the first end of the rehabilitative tape 4. In a fourth step 53 of the method 51, the rehabilitative tape is pushed past the cutting mechanism 9, until the first end 45 extends out of the vertical cutting mechanism 7, as shown in FIG. 3A, and described in associated text herein. In a fifth step 59 of the method 51, the rehabilitative tape is pulled in the direction of arrow 59 to line 149-149, demarking the beginning of the split portion 160, and aligned with the plurality of blades 11 until the second end 47 of the split rehabilitative tape is coextensive with the line 149-149, demarking the beginning of the split portion 160 between lines 149-149 and lines 60-60 of the split portion 160 of split tape 87, shown in FIG. 3C, and described in associated text herein.

In a sixth step 61 of the method 51, one or more of the plurality of blades 11 are set from the non-cutting position 23 to the cutting position 21, such that the exposed length 43 of rehabilitative tape is split into two or more sections 39. In a seventh step 63 of the method 51, the length of the rehabilitative tape 43 is pulled by hand in the direction of the arrow 63 until the first end 45 of the rehabilitative tape is coextensive with the line 60-60, demarking the end of the split portion 160 between lines 60-60 and of the segment of split tape 87, shown in FIG. 3C, and described in associated text herein. In an eighth step 65 of the method 51, the length of rehabilitative tape 43 is pulled by hand in the direction of the arrow 63 until the first end 45 of the split rehabilitative tape is coextensive with the line 70-70, demarking the desired length 37 of the segment of split tape 87, shown in FIG. 3C, and described in associated text herein.

In a ninth step 65 of the method 51, the cutting die punch mechanism 9 is activated, rounding the corners 35 of the second end 47 of the rehabilitative tape and separating it from the remaining portion 51 of the rehabilitative tape roll. In a tenth step 67 of the method 51, the tape is pulled until the second end 47 has been split into two or more sections by the plurality of blades. In an eleventh step 69 of the method 51, the plurality of blades 11 are retracted. In a twelfth step 71 of the method 51, the improved rehabilitative tape is applied to a patient to increase blood circulation and support muscles in an affected area to a patient in need of treatment thereof. In a thirteenth step 72 of the method 51, for future uses of the rehabilitative tape from the same roll, a first end 45 of the rehabilitative elastic tape is currently in cutting die punch mechanism 9, first end 45 is fed into the vertical cutting mechanism 7, until the first end 45 extends out of the vertical cutting mechanism 7. In a fourteenth step 73 of the method 51, steps 53-71 are repeated.

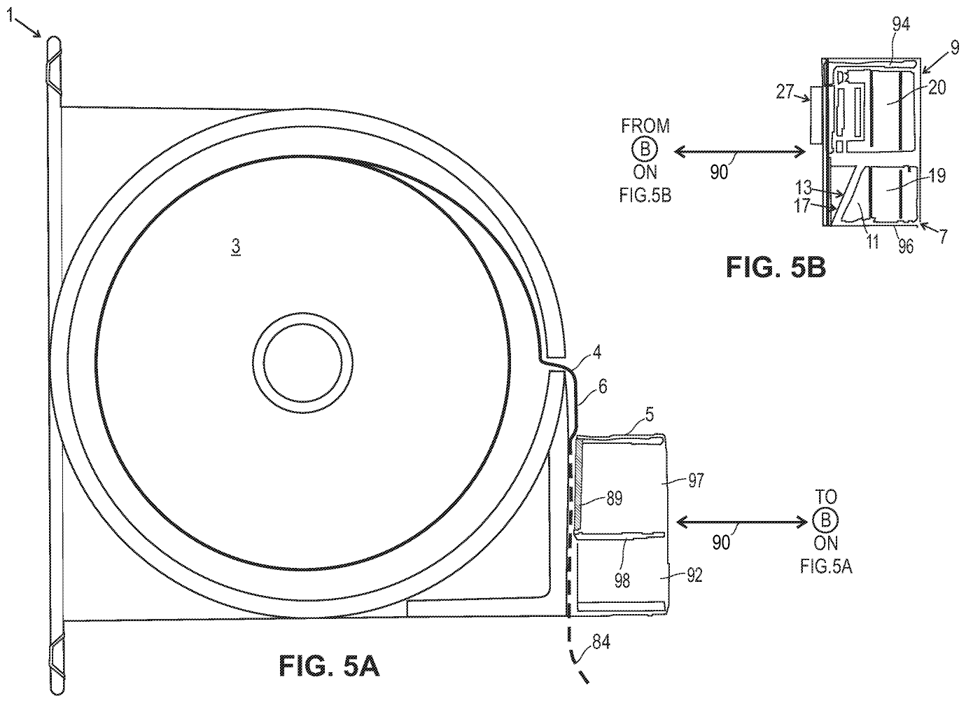

FIGS. 3A-3C depict an Illustrative Example 1 for forming a tape segment 84, depicted in FIG. 5A, using device 1. Specifically, FIG. 3C depicts a cross-sectional view of split tape segment 87, having a desired length 37 and split portion 137A having a desired length 137.

### Illustrative Example 1

Illustrative Example 1 describes using device 1 to produce split tape segment 87 that is an illustrative example of the tape segment 84 from tape 4 shown in FIG. 5A. Referring to FIGS. 3A-3C and the fifth step 59 of the method 51, the rehabilitative tape is pulled in the direction of arrow 59 to line 149-149, demarking the beginning of the split portion 160, and aligned with the plurality of blades 11 until the second end 47 of the split rehabilitative tape is coextensive with the line 149-149, demarking the beginning of the split portion 160 between lines 149-149 and lines 60-60 of the split portion 160 of split tape 87, shown in FIG. 3C, and described in associated text herein.

A length 137 is chosen based on the desired length of the split portion 160. The length 137 may be between 1 inch and 3 ft. Once the desired length 137 of the split portion 160 is established, a position of the line 149-149 may be determined as the straight line perpendicular offset distance from center line 50-50 of the die punch 25 of the cutting die punch mechanism 9 that is equal to the desired length 137 of the split portion 160, as shown in FIG. 3C.

Referring to FIGS. 3A-3C and the seventh step 63 of the method 51, the length of the rehabilitative tape 43 is pulled by hand in the direction of the arrow 63 until the first end 45 of the rehabilitative tape is coextensive with the line 70-70, demarking the end of the split portion 160 between lines 70-70 and the end 47 of the segment of split tape 87, shown in FIG. 3C, and described in associated text herein.

A length 37 is chosen based on the desired length of the split tape 87. The length 37 may be between 1 inch and 3 ft. Once the desired length 37 of the split tape 87 is established, a position of the line 70-70 may be determined as the straight line perpendicular offset distance from center line 50-50 of the die punch 25 of the cutting die punch mechanism 9 that is equal to the desired length 37 of the split tape 87, as shown in FIG. 3C.

In an embodiment, the actuating mechanism is comprised of two or more cutting dies adapted to round the corners of each section of rehabilitative tape.

In an embodiment, the method further comprises operably coupling the cover to a flat surface before operating.

In an embodiment, the method further comprises removing the cover from the flat surface after operating.

In an embodiment, the method further comprises measuring the exposed length of rehabilitative tape against a line demarcated on the cover, until the exposed length of rehabilitative tape has reached an advantageous length for increasing blood circulation in an affected area.

In an embodiment, the method further comprises disposing of worn cutting implements and replacing them with sharp cutting implements when needed. In an embodiment, the disposable cutting implements are selected from the group consisting of single edge razor blades, snap-off blades, and utility knife blades.

In an embodiment, the method further comprises removing the cutting punch for cleaning, and replacing the cutting punch in the cover once cleaned.

In an embodiment, the apparatus allows the plurality of cutting implements 11 to cut the rehabilitative tape into sections from the following list: two halves, three thirds, four quarters, one half and two quarters, One half one sixth and one third, and two quarters two twelfths and two sixths.

In an embodiment, there are five cutting implements. In one embodiment, the implements are positioned at ¼, ⅓, ½, ⅔, and ¾ of the width of the rehabilitative tape.

In an embodiment, the method further comprises five cutting implements.

In an embodiment, the method further comprises the implements positioned at ¼, ⅓, ½, ⅔, and ¾ of the width of the rehabilitative tape.

In an embodiment, the method further comprises the apparatus being usable when operated with only one hand.

In an embodiment, a solvent suitable for dissolving the adhesive commonly used in rehabilitative tape is provided to clean the cutting punch.

In an embodiment, the method further comprises using a solvent suitable for dissolving the adhesive commonly used in rehabilitative tape to clean the cutting punch.

FIGS. 5A and 5B depicts a cross-sectional side view of the rehabilitative tape splitter, in accordance with embodiments of the present invention. In an embodiment, the plurality of blades 11, cutting die 7, punch mechanism 9, die punch 25, or cutting die 27 are replaceably disposable, as a cutting die punch mechanism cartridge 94 and a vertical cutting mechanism cartridge 96. The plurality of blades 11, cutting die 7, punch mechanism 9, die punch 25, or cutting die 27 are insertable and removable along the bi-direction of arrow 91. A compressible bumper 89 comes in contact with the tape when the cutters (9, 7) are pressed into cutting position 21 and provides tension on the tape during cutting when it is cut into strips. The bumper is rubber or something similar that will grip the tape during cutting.

In FIG. 5A, a portion of tape 4 (denoted by a dashed line) is fed into the cutting die chamber 97 after insertion of the cartridges 94 and 96 in a direction shown by a bi-directional arrow 90, enabling the cartridges 94, 96 and chambers 97 and 92 to mate. A bumper 89 of the cutting die chamber 97 is proximal to the surface 6 of the tape 4, shown by hatching in FIG. 5A. The bumper 89, provides a compressible layer between the bottom of cutting die chamber 97 and a surface 6 of the tape 4. The bumper 89 is pushed against the surface 6 of the tape 4 when the die cutting punch 25 Is in cutting position 21. This pushing increases the tension on the surface 6 of the tape 4 during cutting, such that the bumper 89 grips the tape during cutting. The bumper 89 is made of rubber or synthetic elastomers like silicone. It can be compressible, so it can hold and position the tape 4 in the die 27 during cutting. The bumper 89 is only between cutting die 27 and the die bunch 25.

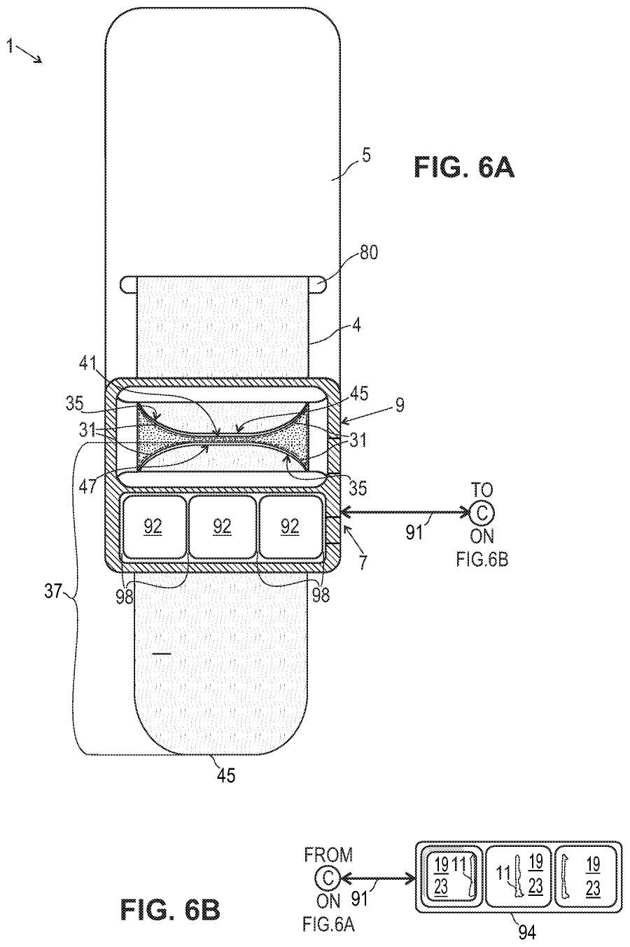

In an embodiment, the vertical cutting mechanism 7 has wall partitions 98, ensuring that the plurality of blades 11 is spaced sufficiently to create usable splits of tape. In an embodiment, the disposable cutting implements are selected from the group consisting of single edge razor blades, snap-off blades, and utility knife blades. FIGS. 6A and 6B depicts a cross-sectional front view of the rehabilitative tape splitter, in accordance with embodiments of the present invention. In an embodiment, the cutting punch 9 and vertical cutting mechanisms 7 are removable for cleaning. The cutting punch 9 and vertical cutting mechanisms 7 are insertable and removable along the direction of bi-directional arrow 91. FIG. 6A shows die chamber 97 and cutting mechanism cavities 92 are left in the housing 5 when the cutting mechanisms 9, 7 have been removed. Combined FIGS. 6A-6B show the die cutting mechanism 9 and the vertical cutting mechanism 7 may be inserted into their respective cavities 97, 92 by inserting them in the direction of the arrows 90, 91, 100, and 101. The cutting mechanisms, 9, 7 may be held in place by frictional force generated by the natural tendency of the compressible bumper to expand against the force of compression arising from insertion of the cutting mechanisms 9, 7 that are slightly longer and wider than the corresponding dimensions of their respective cavities 97, 92.

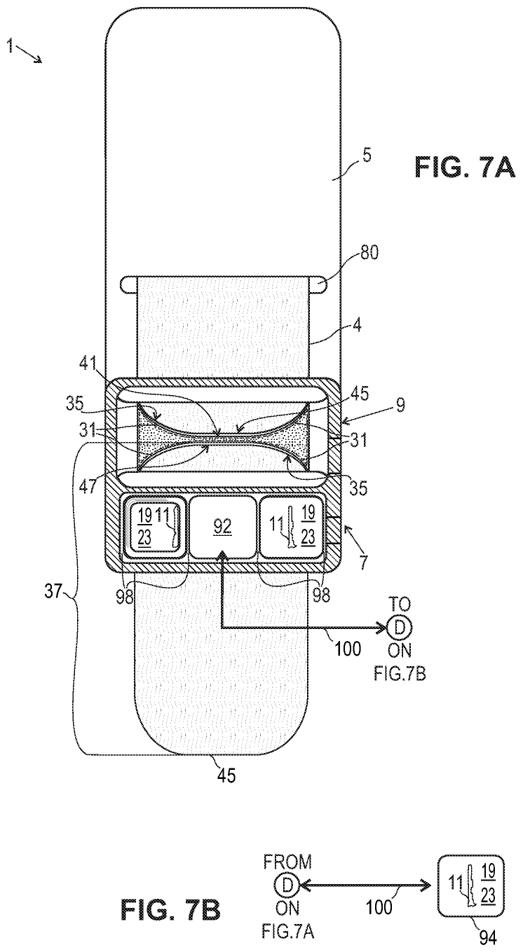

FIGS. 7A and 7B depicts a cross-sectional front view of the rehabilitative tape splitter, in accordance with embodiments of the present invention. In an embodiment, the cutting punch 9 and vertical cutting mechanisms 7 are removable for cleaning. The cutting punch 9 and vertical cutting mechanisms 7 are insertable and removable along the direction of bi-directional arrow 100.

FIG. 7A shows a cavity is left when cutting mechanism 9 has been removed form housing 5. Combined FIGS. 7A-7B show the vertical cutting cartridge 94 may be inserted into the cavity 92 by inserting it in the direction of arrow 100.

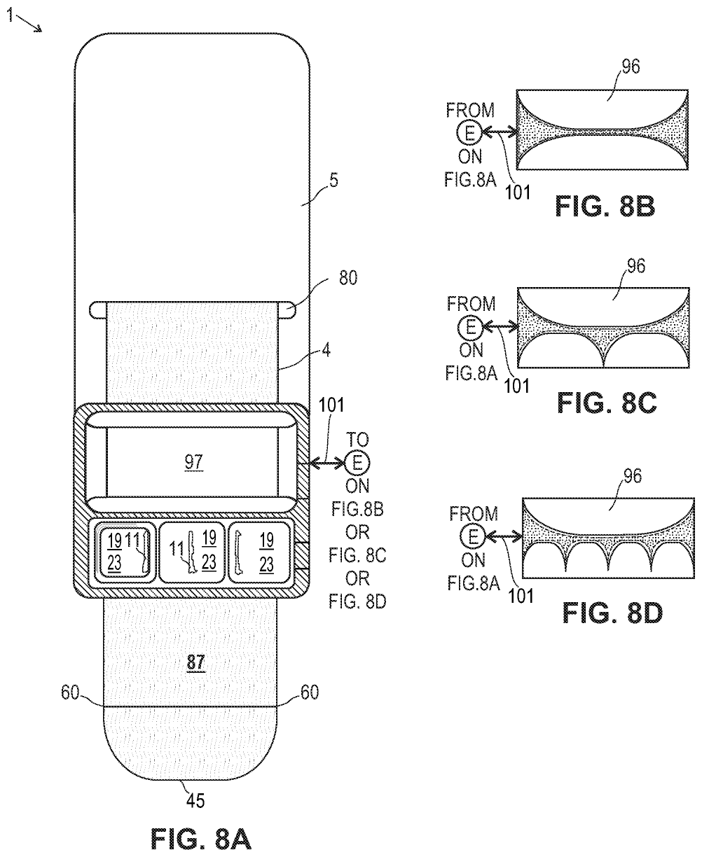

FIGS. 8A, 8B, 8C, and 8D depicts a cross-sectional front view of the rehabilitative tape splitter, in accordance with embodiments of the present invention. In an embodiment, the cutting punch 9 and vertical cutting mechanisms 7 are removable for cleaning or replacement. The cutting punch 9 and vertical cutting mechanisms 7 are insertable and removable along the direction of bi-directional arrow 101.

FIG. 8A shows cutting die chamber 97 is left empty when a die cutter cartridge 94 has been removed from cutting die chamber 97. Combined FIGS. 8A-8B show the blade die cutter cartridge 94 may be inserted into the cutting die chamber 97 by inserting it in the direction of bi-directional arrow 101.

FIGS. 8A and 8B show unsplit tape segment 77 being formed when die cutter cartridge 96 having die punch 25 for forming rounding corners 35 at each corner of unsplit tape segment 77, as shown in FIG. 8B, has been inserted in die cutter chamber 97.

FIG. 5 FIGS. 6A-6B, 7A-7B, and 8A-8B, show replacable cutting mechanisms as module (9, 7) or individual cutting blades 11 within the modules.

FIG. 4 depicts a flow diagram listing the steps of a method 51 of cutting rehabilitative tape. The method 51 includes a step 46, providing a hand-held and operated device 1 for splitting rehabilitative elastic tape into segments and rounding corners of the rehabilitative elastic tape segments. The device is comprised of a feed roll 3 of rehabilitative elastic tape 4, having a substantially uniform thickness and width, and a housing 5. The housing 5 is comprised of a vertical cutting mechanism 7 and a cutting die punch mechanism 9 operably coupled in tandem. The vertical cutting mechanism is comprised of a plurality of blades 11. Each blade has a cutting edge 13.

There are a plurality of actuator buttons 19 operably coupled to and aligned with the blades 11 for setting the blades 11 to either a cutting position 21 or a non-cutting position 23, such that when the blades 11 are in the cutting position 21 the cutting edge 13 is in contact with surface 6 of the tape 4. The cutting implements in the cutting position 21 splits the rehabilitative tape 4 into two or more sections 39. The cutting die punch 9 mechanism is comprised of a pair of mating tools, a die punch 25 and a cutting die 27. The die punch 25 is provided with a fixed punching blade member 29 and the cutting die 27 with a flexible cutting blade member 31, with the fixed punching blade member 29 and said flexible cutting blade member 31 each having a continuous cutting edge 33. The die punch is adapted to be moved into the cutting die by hand and attain the closest possible mating of said members 29, 31 for cutting or forming the rounded corners 35 of a tape segment 37 when the tape is fed into the punching mechanism.

FIGS. 3A, 3B, and 3C depict a segment of split tape, in accordance with embodiments of the present invention. The rehabilitative tape has a first end 45, a second end 47, a splitting point 49 and a remaining portion 51. The first step 53 of the method 51 is stretching a length of rehabilitative tape from the tape roll, placing the first end 45 in the cutting die punch mechanism 9. Then, the second step 57 of the method is activating the cutting die punch mechanism 9, and rounding the corners 35 of the first end 45 of the rehabilitative tape 4. Then, the third step 59 of the method is pulling the rehabilitative tape to the splitting point 49, aligned with the plurality of blades 11. The fourth step 61 of the method is setting one or more of the plurality of blades 11 from the non-cutting position 23 to the cutting position 21 so that the exposed length 43 of rehabilitative tape is split into two or more sections 39. The fifth step 63 of the method is pulling the length of rehabilitative tape 43 by hand until the split rehabilitative tape until the second end 47 is aligned with the cutting die punch mechanism 9. The sixth step 65 of the method is activating the cutting die punch mechanism 9, rounding the corners 35 of the second end of the rehabilitative tape and separating it from the remaining portion 51 of the rehabilitative tape roll. The seventh step 67 of the method is pulling the tape until the second end 47 has been split into two or more sections by the plurality of blades. The eighth step 69 of the method is retracting the plurality of blades. The ninth step 71 of the method is applying the improved rehabilitative tape to a patient to increase blood circulation and support muscles in an affected area.

In an embodiment, the actuating mechanism is comprised of two or more cutting dies adapted to round the corners of each section of rehabilitative tape.

In an embodiment, the method further comprises operably coupling the cover to a flat surface before operating.

In an embodiment, the method further comprises removing the cover from the flat surface after operating.

In an embodiment, the method further comprises measuring the exposed length of rehabilitative tape against a line demarcated on the cover, until the exposed length of rehabilitative tape has reached an advantageous length for increasing blood circulation in an affected area.

In an embodiment, the method further comprises disposing of worn cutting implements and replacing them with sharp cutting implements when needed. In an embodiment, the disposable cutting implements are selected from the group consisting of single edge razor blades, snap-off blades, and utility knife blades.

In an embodiment, the method further comprises removing the cutting punch for cleaning, and replacing the cutting punch in the cover once cleaned.

In an embodiment, the apparatus allows the plurality of cutting implements 11 to cut the rehabilitative tape into sections from the following list: two halves, three thirds, four quarters, one half and two quarters, One half one sixth and one third, and two quarters two twelfths and two sixths.

In an embodiment, there are five cutting implements. In one embodiment, the implements are positioned at ¼, ⅓, ½, ⅔, and ¾ of the width of the rehabilitative tape.

In an embodiment, the method further comprises five cutting implements.

In an embodiment, the method further comprises the implements positioned at ¼, ⅓, ½, ⅔, and ¾ of the width of the rehabilitative tape.

In an embodiment, the method further comprises the apparatus being usable when operated with only one hand.

In an embodiment, a solvent suitable for dissolving the adhesive commonly used in rehabilitative tape is provided to clean the cutting punch.

In an embodiment, the method further comprises using a solvent suitable for dissolving the adhesive commonly used in rehabilitative tape to clean the cutting punch.

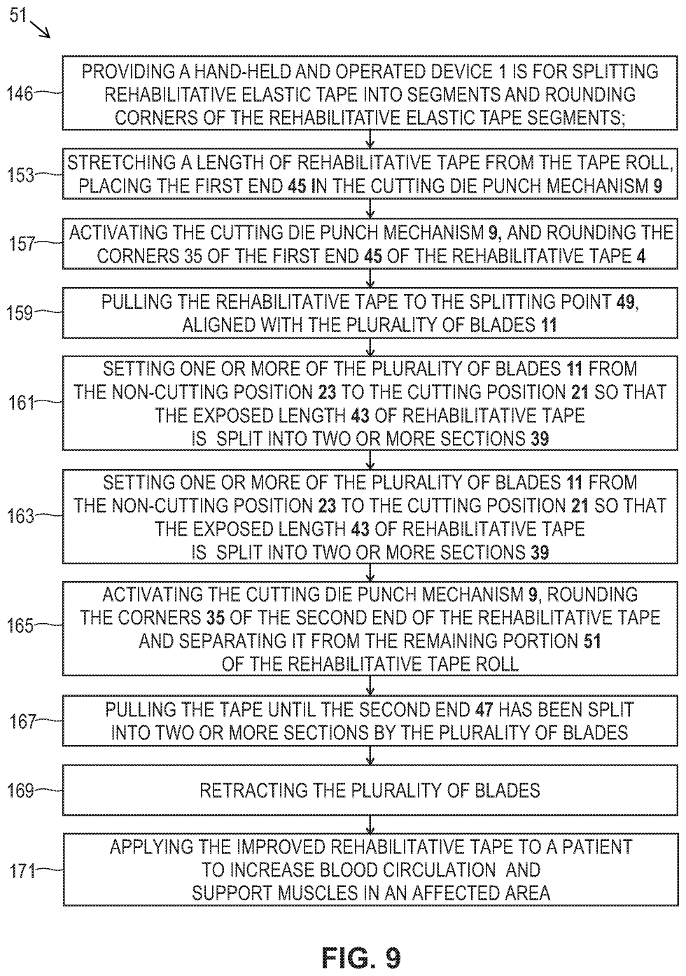

FIG. 9 depicts a flow diagram of a method 151 of cutting rehabilitative tape 4. An illustrative example of forming of a segment of split tape 87, by cutting rehabilitative tape, is shown in FIGS. 3A-3C, and described in associated text herein. In a first step 146 of the method 51, a hand-held and operated device 1 is provided for splitting rehabilitative elastic tape into segments and rounding corners of the rehabilitative elastic tape segments. In a second step of the method 151 includes the hand-held and operated device 1 for splitting rehabilitative elastic tape 4 into segments 37, 87 and rounding corners 35. In a second step 153 of the method 51 a length of rehabilitative tape is stretched from the tape roll, placing the first end 45 in the cutting die punch mechanism 9. In a third step 157 of the method 151 the cutting die punch mechanism 9 is activated, rounding the corners 35 of the first end 45 of the rehabilitative tape 4. In a fourth step 159 of the method 151, the rehabilitative tape is pulled to the splitting portion 49, and aligned with the plurality of blades 11. In a fourth step 161 of the method 151 is setting one or more of the plurality of blades 11 from the non-cutting position 23 to the cutting position 21 so that the exposed length 43 of rehabilitative tape is split into two or more sections 39. In a fifth step 163 of the method 151 a length of rehabilitative tape 43 is pulled by hand until the second end 47 is aligned with the cutting die punch mechanism 9. In a sixth step 165 of the method is activating the cutting die punch mechanism 9, rounding the corners 35 of the second end of the rehabilitative tape and separating it from the remaining portion 51 of the rehabilitative tape roll. The seventh step 67 of the method is pulling the tape until the second end 47 has been split into two or more sections by the plurality of blades. In an eighth step 169 of the method 161, the plurality of blades 11 is retracted. In a ninth step 171 of the method the improved rehabilitative tape is applied to a patient for a specific manual therapy application.

While exemplary embodiments have been specifically disclosed, it should be understood that the practice of this invention is not limited to those embodiments. Modifications and variations falling within the spirit of the invention will occur to those skilled in the art. Therefore, it is not intended that the scope of the invention be determined by the disclosed exemplary embodiments, but rather should be determined by the breadth of the appended claims.

1. A hand-held and operated device for splitting rehabilitative elastic tape into segments and rounding corners of the rehabilitative elastic tape segments, comprising:
a feed roll of rehabilitative elastic tape, having a substantially uniform thickness and width;
a housing (5), comprising:
a vertical cutting mechanism (7) and a cutting die punch mechanism (9) operably coupled in tandem, the vertical cutting mechanism (7), comprising:
a plurality of blades (11),
wherein each blade (11) has a cutting edge (13),
wherein the cutting edge (13) of the blade extends in the opposite direction of the relative motion between the tape (4) and the blades (11) when the tape (4) is fed into the vertical cutting mechanism (7),
wherein the angle (17) between the cutting edge (13) of the planar blades and the surface of the tape is between 15 and 75 degrees;
a plurality of actuator buttons operably coupled to and aligned with the blades for setting the blades to either a cutting position or a non-cutting position (23), such that when the blades are in the cutting position the cutting edge is in contact with surface of the tape, and
the cutting die punch mechanism (9), comprising:
a pair of mating tools, a die punch (25) and a cutting die (27),
characterized in that said die punch (25) is provided with a fixed punching blade member (29) and said cutting die (27) with a flexible cutting blade member (31), said fixed punching blade member (29) and said flexible cutting blade member (31) each having a continuous cutting edge, said die punch (25) being adapted to be moved into said cutting die (27) by hand and attain a closest possible mating of said members for cutting or forming the rounded corners (35) of a tape segment (37) when the tape (4) is fed into the die cutting punch mechanism (9).

2. A hand-held and operated device for splitting rehabilitative elastic tape into segments and rounding corners of the rehabilitative elastic tape segments, comprising:
a feed roll of rehabilitative elastic tape, having a substantially uniform thickness and width;
a housing, comprising:
a vertical cutting mechanism and a cutting die punch mechanism operably coupled in tandem, the vertical cutting mechanism, comprising:
a plurality of blades,
wherein each blade has a cutting edge;
a plurality of actuator buttons operably coupled to and aligned with the blades for setting the blades to either a cutting position or a non-cutting position, such that when the blades are in the cutting position the cutting edge is in contact with surface of the tape,
wherein the cutting implements in the cutting position split the rehabilitative tape into two or more sections,
the cutting die punch mechanism, comprising:
a pair of mating tools, a die punch and a cutting die, characterized in that said die punch is provided with a fixed punching blade member and said cutting die with a flexible cutting blade member, said fixed punching blade member and said flexible cutting blade member each having a continuous cutting edge, said die punch being adapted to be moved into said cutting die by hand, attaining a closest possible mating of said members for cutting or forming the rounded corners of a tape segment when the tape is fed into the punching mechanism.

3. The apparatus of claim 2 wherein the die punch is in the shape of two oppositely aligned half circles connected at the point 90 degrees along their arc, such that a single die punch is able to round the corners of incoming and outgoing pieces of tape.

4. The apparatus of claim 2 wherein there are a plurality of die punches each one designed to round the corners of a specific section of tape, sections of tape selected from the group consisting essentially of a whole piece of tape, a half piece of tape, a third of a piece of tape, a fourth of a piece of tape, a sixth of a piece of tape, and a twelfth of a piece of tape.

5. The apparatus of claim 2, wherein the apparatus is operably coupled to a flat surface.

6. (canceled)

7. The apparatus of claim 2, wherein the exposed length of rehabilitative tape is demarcated to an advantageous length for increasing blood circulation in an affected area.

8. The apparatus of claim 2, wherein the plurality of blades, cutting die, punch mechanism, die punch, or cutting die are replacably disposable.

9. The apparatus of claim 8, wherein the disposable cutting implements are selected from the group consisting of single edge razor blades, snap-off blades, and utility knife blades.

10. The apparatus of claim 2, wherein the cutting punch is removable for cleaning.

11. A method of splitting rehabilitative elastic tape into sections (39), comprising:
providing a hand-held and operated device for splitting rehabilitative elastic tape into sections (39) and rounding corners (35) of the rehabilitative elastic tape sections, comprising:
a feed roll of rehabilitative elastic tape, having a substantially uniform thickness and width;
a housing, comprising:
a vertical cutting mechanism and a cutting die punch mechanism operably coupled in tandem, the vertical cutting mechanism, comprising:
a plurality of blades,
wherein each blade has a cutting edge, a plurality of actuator buttons operably coupled to and aligned with the blades for setting the blades to either a cutting position or a non-cutting position, such that when the blades are in the cutting position the cutting edge is in contact with a surface of the tape, and
wherein the cutting implements in the cutting position split the rehabilitative tape into two or more sections (39),
the cutting die punch mechanism, comprising:
a pair of mating tools, a die punch and a cutting die, characterized in that said die punch is provided with a fixed punching blade member and said cutting die with a flexible cutting blade member, said fixed punching blade member and said flexible cutting blade member each having a continuous cutting edge, said die punch being adapted to be moved to operably couple said cutting die by hand, attaining a closest possible mating of said members for cutting or forming the rounded corners of a tape segment when the tape is fed into the punching mechanism,
wherein the rehabilitative tape has a first end (45), a second end (47), a splitting point (49) of tape (4) and a remaining portion (51) of tape (4);
stretching a length of rehabilitative tape from the tape roll, placing a first end (45) in the cutting die punch mechanism (9);
activating the cutting die punch mechanism, rounding the corners of the first end (45) of the rehabilitative tape (4);
pulling the rehabilitative tape to the splitting point, aligned with the plurality of blades;
setting one or more of the plurality of blades from the non-cutting position to the cutting position so that the exposed length of rehabilitative tape is split into two or more sections;
pulling the length of rehabilitative tape by hand until the split rehabilitative tape until the second end is aligned with the cutting die punch mechanism;
activating the cutting die punch mechanism, rounding the corners of the second end of the rehabilitative tape and separating it from the remaining portion of the rehabilitative tape roll;
pulling the tape until the second end has been split into two or more sections by the plurality of blades;
retracting the plurality of blades; and
applying the improved rehabilitative tape to a patient to increase blood circulation, and support muscles in an affected area needing support.

12. The method of claim 11, wherein the actuating mechanism is comprised of two or more cutting dies adapted to round the corners of each section of rehabilitative tape.

13. The method of claim 11, comprising operably coupling the cover to a flat surface.

14. The method of claim 13, wherein the apparatus is removed from the flat surface once the multiple sections of rehabilitative tape have been removed from the cover.

15. The method of claim 11, comprising measuring the exposed length of rehabilitative tape against a line demarcated on the cover, until the exposed length of rehabilitative tape has reached an advantageous length for increasing blood circulation in an affected area.

16-17. (canceled)

18. The method of claim 11, comprising removing the cutting punch for cleaning, and replacing the cutting punch in the cover once cleaned.

19. (canceled)

20. The method of claim 11, wherein the plurality of cutting implements are arranged to cut the rehabilitative tape into sections from the following list two halves, three thirds, four quarters, one half and two quarters, One half one sixth and one third, and two quarters two twelfths and two sixths.

21-24. (canceled)

25. The method of claim 11 wherein the apparatus can be used with only one hand.

26-28. (canceled)

29. The apparatus of claim 2, wherein the cutting edge (13) of the blade extends in the opposite direction of the relative motion between the tape (4) and the blades (11) when the tape (4) is fed into the vertical cutting mechanism (7).

30. The apparatus of claim 2, wherein the angle (17) between the cutting edge (13) of the planar blades and surface of the tape is between 15 and 75 degrees.

31. The apparatus of claim 2 wherein there are 5 cutting implements.

32. The apparatus of claim 21, wherein there are 5 cutting implements positioned at ¼, ⅓, ½, ⅔, and ¾ of the width of the rehabilitative tape.
